(19) United States
(12) Patent Application Publication
(10) Pub. No.: US 2004/0092853 A1
(43) Pub. Date: May 13, 2004

(54) Orthopaedic splint

(76) Inventors: Michael Degun, Essex (GB); Iain Andrew Craig Lennox, Essex (GB)

Correspondence Address: Gunnar G Leinberg, Nixon Peabody, Clinton Square, PO Box 31051, Rochester, NY, 14603-1051, US

(21) Appl. No.: 10/471,536

(22) PCT Filed: Mar. 13, 2002
(86) PCT No.: PCT/GB02/01154

(30) Foreign Application Priority Data
Mar. 13, 2001 (GB) 0106111.8

(51) Int. Cl. A61F 5/00

(52) U.S. Cl. 602/27

(57) ABSTRACT

An orthopaedic splint (1) comprises at least one splint member (2, 3) adapted to be secured to a limb such as a lower arm and wrist. The splint member (2, 3) is associated with an inflatable air-bag (6) disposed, in use, between the splint member (2, 3) and the limb. The air-bag (6) is fitted with a valve or valves (7) by which the air-bag (6) can be inflated and by which air is released from within the air-bag (6) if the pressure of air within the air-bag (6) exceeds a predetermined threshold.

This invention relates to improvements in orthopaedic splints, and in particular to improvements in pneumatic splints of the type comprising rigid members associated with one or more liners which are, in use, inflated to engage the body part to which the splint is applied.

In the present context, the term “splint” is intended to mean a rigid assembly for application to a body part, most commonly a limb, so as to support and maintain parts of the limb in a constant mutual disposition, thereby facilitating repair of a fracture or other tissue damage.

An orthopaedic splint of the general type with which the present invention is concerned, intended for application to an ankle, is disclosed in U.S. Pat. No. 3,955,565. Such a splint comprises a pair of rigid shell members which cooperate to define a shell that can be mounted about an ankle. The two shell members are fitted with touch-and-close (VELCRO-type) fasteners by which they can be held together and in position. Inflatable air-bags are disposed within the shell members such that when inflated the air-bags conform to and engage the lower limb. Each air-bag is fitted with a valve by which air may be introduced into the air-bag, but is prevented from escaping.

Developments of the device described above are disclosed in U.S. Pat. No. 4,628,945, U.S. Pat. No. 5,125,400 and European Patent No 0252121. U.S. Pat. No. 4,287,920 describes a form of self-sealing valve that can be used in such devices. This valve has a mouth, the internal surfaces of which are coated with a tacky, grease-like substance. This causes the juxtaposed surfaces to adhere to each other, thereby sealing the mouth of the valve in a leak-proof manner.

There has now been devised an improvement to devices of the general type described above.

According to the invention, an orthopaedic splint comprises at least one rigid member adapted to be secured to a part of the human or animal body to provide support thereto, the rigid member being associated with an inflatable air-bag disposed, in use, between the rigid member and the body part to which the splint is applied, the air-bag being fitted with valve means by which air can be introduced into the air-bag and retained therewithin and by which air is released from within the air-bag if the pressure of air within the air-bag exceeds a predetermined threshold.

The splint according to the invention is advantageous primarily in that it cannot be inflated to a pressure in excess of the predetermined threshold. It has been found that such excessive pressure can arise in pneumatic splints of the type known in the prior art, as a result either of over-inflation when the device is applied to the body, or as a result of oedema (swelling) that occurs subsequently. Thus, the splint can be applied and inflated until it has a tight fit with the body part. If, subsequent to fitting, the swelling in the patient's limb were to decrease, then the inflation can be “topped up”, either by a medical practitioner or more commonly by the patient himself, so that tight engagement of the splint with the limb is maintained. However, if the swelling of the limb were to increase, as is commonly the case, the air-bag will automatically deflate such that the pressure remains at the predetermined threshold. The application of excessive pressure to the limb, which could have the consequence of inhibiting or cutting off the supply of blood to the limb, is thereby avoided. The splint according to the invention is therefore able to hold the injured limb tightly, even if the patient's swelling decreases, yet is safe in that it cannot be fitted too tightly. Even if further swelling occurs after fitting, the splint cannot become too tight, as it will self-deflate.

The valve means may comprise a single valve which is used for both inflation of the air-bag and release of pressure in the event that the internal pressure rises above the pre-determined threshold. However, it is preferred that separate valves be provided for inflation (such a valve being, for instance, similar to the type of valve used in a prior art pneumatic splint) and for pressure-release, though these separate valves may be integrated into a single unit.

Valves serving for both inflation and release of excessive pressure may be similar to types supplied by Halkey-Roberts Corporation of 11600 9th Street North, St Petersburg, Fla. 33716, USA. A particular valve suitable for inflation of the splint is the valve supplied by that company under the product number C2472000xx. The pressure relief valve may be similar to that supplied by Halkey-Roberts under the product number C24781xxxx.

Apart from the valve used for pressure relief, the device according to the invention may be generally similar to prior art devices. It may comprise one or more rigid splint members, which are most conveniently formed, eg by moulding, from suitably rigid synthetic plastics materials.

Most preferably, the splint according to the invention comprises a pair of cooperating splint members, each of generally hemi-cylindrical form, the two splint members together defining a generally tubular casing that can be fitted about a limb or part of a limb.

Where the splint according to the invention comprises more than one rigid member, the rigid members are preferably secured together by releasable fastenings. “Touch-and-close” or “hook-and-loop” (VELCRO-type) fasteners are particularly suitable for this purpose, and at least one (and more preferably several) such fasteners are therefore preferably affixed to the rigid members that are to be secured together. Such fasteners normally take the form of tapes having mating male and female components.

A single air-bag may be incorporated into the device, or there may be separate airbags associated with some or all of several rigid splint members. There may also be more than one air-bag associated with a single splint member. Where there is more than one air-bag, each air-bag is preferably provided with valve means in accordance with the invention.

In a currently preferred embodiment, a single air-bag is provided, the air-bag having the form of a plurality of tentacles or fingers connected at one end thereof and extending longitudinally along the interior of each of two splint members that together form, in use, a generally tubular casing.

The air-bag may be of any suitably flexible and air-tight material. Plastics materials are again preferred. Most preferably, the material from which the air-bag is formed is elastic. This facilitates conformance of the inflated air-bag to the shape of the limb to which the splint is applied and the accommodation of changes in the form of the limb as it swells and then returns to normal.

The air-bag may bear directly upon the patient's skin. Preferably, however, an intermediate material, eg a soft fabric or foam material, may be interposed between the air-bag and the patient. As well as enhancing comfort for the patient, such a layer of foam material may provide for a more even distribution of pressure.

A layer of foam material is preferably also interposed between the air-bag and the internal surface of the splint member. The air-bag is thus preferably encased between two layers of foam material, one between the air-bag and the splint member and the other between the air-bag and the patient's skin.

Because the foam material is in constant contact with the patient's skin, it is preferably of a permeable material that allows the skin to “breathe”. One or more openings may also be provided in the splint member to enhance flow of air to and from the patient's skin.

While the description above has focussed principally on pre-formed splints comprising one or more, preferably two or more cooperating components formed, for instance, in plastics material, it will be appreciated that the invention also encompasses rigid splints and the like formed by casting of material directly about an injured limb. In such a case, the air-bag is simply interposed between the limb and the material cast about it. Obviously, it is necessary for the valve means to be accessible to provide for inflation of the air-bag after hardening of the cast and for venting of air from the air-bag in the event that excessive pressure develops.

The splint according to the invention has been described above in terms of its application to a “limb”. It will be apparent to those skilled in the art that appropriately-formed splints according to the invention can be applied to any suitable part of the body. This will most commonly be a limb, or part of a limb such as the ankle, wrist, knee or elbow, but is not necessarily so. The term “limb” as used herein should be interpreted to encompass all such body parts to which the splint may be applied.

The invention will now be described in greater detail, by way of illustration only, with reference to the accompanying drawings, in which

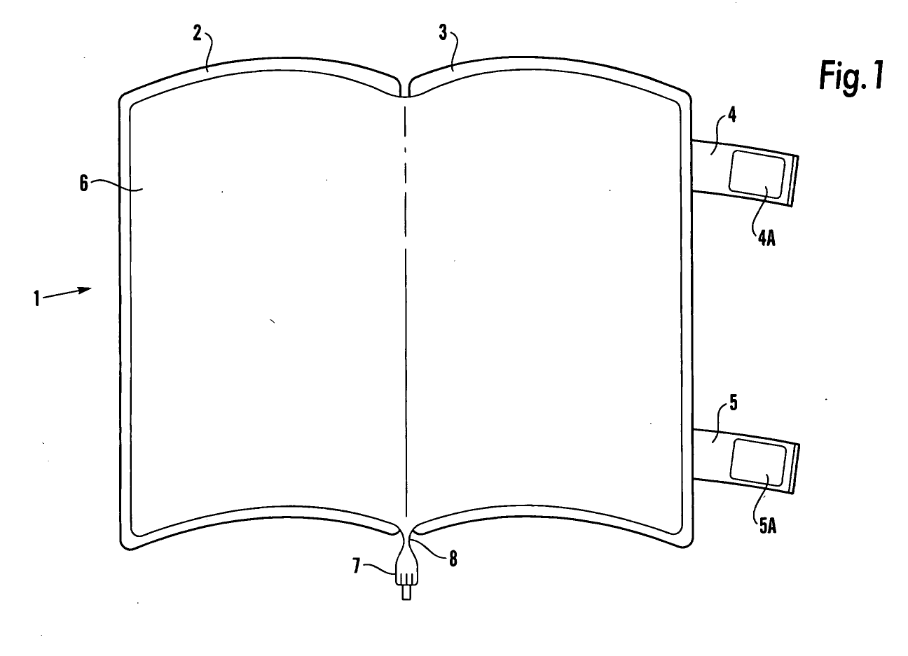

FIG. 1 is an internal view of a first embodiment of a wrist splint in accordance with the invention, in an opened condition prior to use;

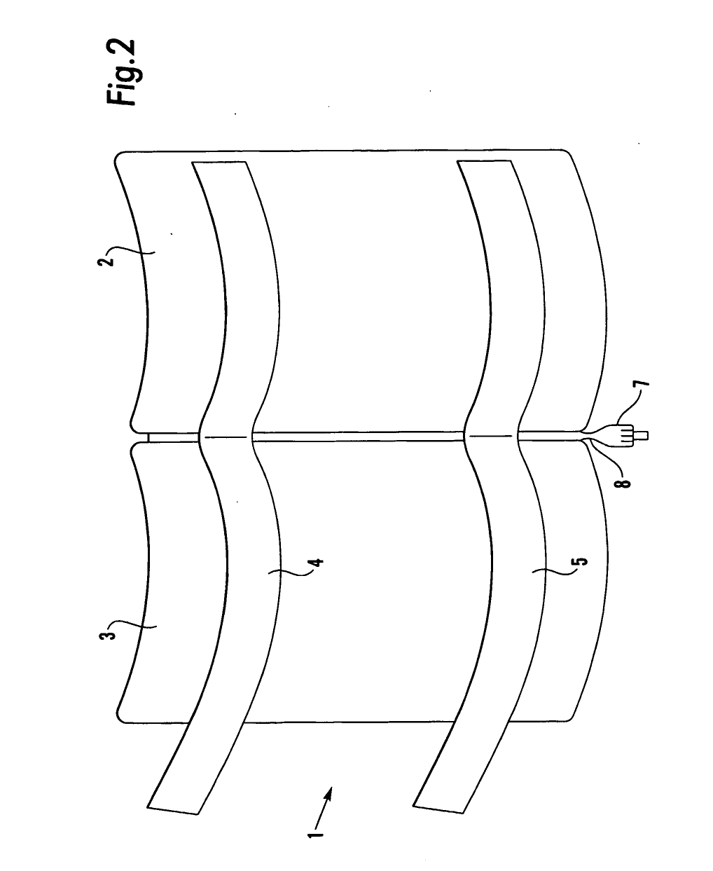

FIG. 2 is view similar to FIG. 1, but showing the exterior of the splint;

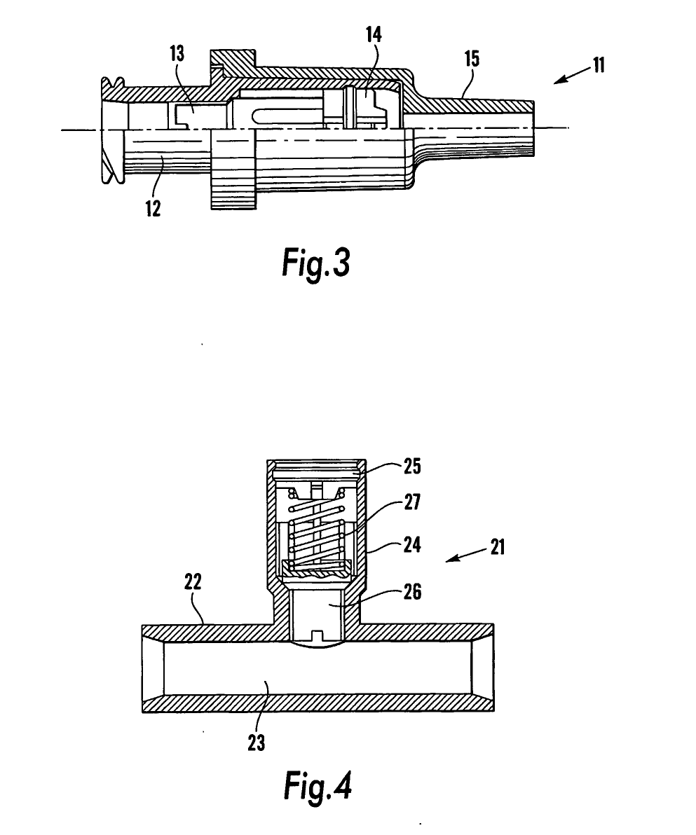

FIG. 3 is a view, partially in section of an inflation valve used in the splint;

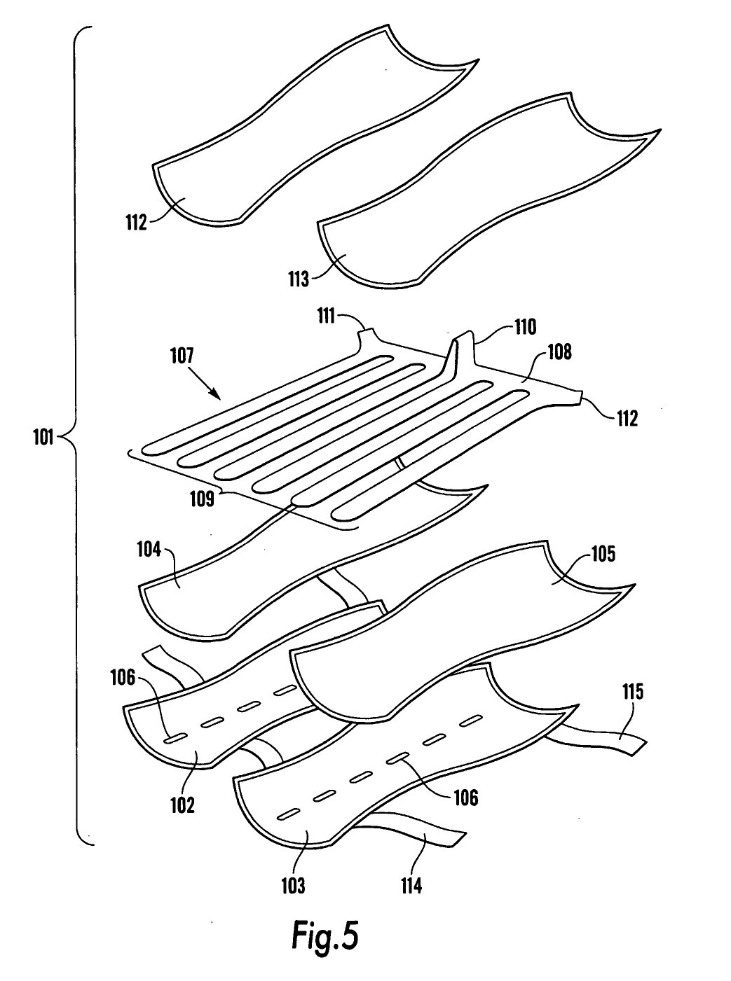

FIG. 4 is a sectional view of a pressure relief valve used in the splint; and

FIG. 5 is an exploded view of a second, currently preferred, embodiment of a wrist splint in accordance with the invention.

Referring first to FIG. 1, a first embodiment of a wrist splint in accordance with the invention is generally designated 1 and comprises a complementary pair of rigid splint members 2,3, each of which is of generally hemi-cyclindrical form and is moulded in plastics material.

The two splint members 2,3 are hingedly connected along one major edge of each by a pair of elasticated tapes 4,5 that pass around the outside of each splint member 4,5 (see FIG. 2) and are bonded to the outside surfaces of the splint members 2,3. The tapes 4,5 extend at one end beyond the edge of the right-hand (as viewed in FIG. 1) edge of the splint member 3. The internally-facing side of the extending part of each tape 4,5 is provided with a patch (4a,5a respectively) of a male (hook) part of a touch-and-close fastener. The external surfaces of the tapes 4,5 have raised loop pile or plush that serves as the female (loop) part of the fastener.

The greater part of the internal surfaces of the splint members 2,3 is covered by an inflatable liner 6 which is fitted with an inflation and pressure-relief valve unit 7 which is mounted, near the hinged spine of the splint 1, on the end of short length of tubing 8 that opens into the liner 6.

The valve unit 7 comprises two valves: an inflation valve 11 (shown in FIG. 3) and a pressure relief valve 21 (see FIG. 4). The inflation valve 11 is similar to that supplied by Halkey-Roberts Corporation under the product number C2472000xx, and comprises a rigid plastics body 12 containing a silicone valve stem 13 and a polypropylene plug 14. The body 12 is fitted with a male luer adapter 15.

The pressure relief valve 21 is similar to Halkey-Roberts type C24781xxxx and comprises a rigid body 22 of T-section, having a principal channel 23 from which a valve limb 24 is upstanding. The valve limb. 23 houses a terminal plug 25 and a valve stem 26 between which a stainless steel compression spring 27 acts with a predetermined pressure (which is set by the characteristics of the spring 27).

The male luer adapter 15 of the inflation valve 11 is inserted into, and bonded to, one end of the principal channel 23 of the pressure relief valve 21 to form the complete valve unit 7. The other end of the principal channel 23 is connected, via the tubing 8, to the liner 6.

In use, the splint 1 is applied to an injured wrist as follows. First, the left-hand (as viewed in FIG. 1) splint member 2 is positioned beneath the wrist and the other splint member 3 then folded over the first splint member 2 so that the two splint members 2,3 together encapsulate the wrist. The enclosure so formed is held together by pulling the ends of the tapes 4,5 around the abutting free edges of the two splint members 2,3 and engaging patches 4a,5a of male hooks with the plush surface of the tapes 4,5 bonded to the splint member 2.

The liner 7 is then inflated by either the fitter or the patient, either using a suitable pump attached to the inflation valve 11 or simply be blowing air through the inflation valve 11, this most conveniently being achieved by attachment to the inflation valve 11 of a tube of sufficient length to reach the patient's (or fitter's) mouth without the wrist having to be raised to a possibly uncomfortable position.

The liner 6 is inflated until the splint 1 has a close and tight fit with the wrist. If a pre-determined pressure (above which blood flow could be impaired) is exceeded, then air is automatically released through the pressure relief valve 21 until the pressure falls to that pre-determined level. Similarly, the pressure is prevented from rising in the event that further swelling of the injured wrist occurs, leading to compression of the inflated liner 6. Nonetheless, if the swelling reduces, the liner 6 can be further inflated very easily, so as to maintain the snug fit of the splint 1 with the wrist.

Turning now to FIG. 5, a second and currently preferred embodiment of a wrist splint according to the invention is generally designated 101 and is shown in exploded view.

The splint 101 comprises a pair of rigid splint members 102,103 which cooperate to form a generally tubular casing for a patient's lower arm and wrist. The splint members 102,103 are moulded in rigid plastics material and are shaped to suit a range of typical arm profiles. Two hook-and-loop fastening straps 114,115 are fixed to the exterior of the two splint members 102,103 so as to hingedly connect the splint members 102,103 together and to enable the splint 101 to be secured to a patient's wrist as described below.

A first pair of foam inserts 104,105 are cut from sheets of permeable foam material and are shaped so as to fit within the splint members 102,103. The foam material is permeable to air, so as to permit flow of air through the foam inserts 104,105. The splint members 102,103 are each formed with a row of openings 106 to facilitate such flow of air.

An air-bag 107 is formed in an elastic, plastics material, and has the form of a manifold, comprising a common terminal portion 108 from which extend six elongate tentacles 109. The tentacles 109 are arranged in two sets of three, the two sets being connected by an umbilical 110 that forms part of the common terminal portion 108 of the air-bag 107. An inflation valve 111 is fitted at one end of the common terminal portion 108, and a pressure relief port 112 is provided at the other end.

The splint 101 is completed by a second pair of foam inserts 112,113 of similar form to the first pair of foam inserts 104,105.

The splint 101 is assembled by first attaching the straps 114,115 to the splint members 102,103. The first pair of foam inserts 104,105 are then fitted into the interior of the respective splint members 102,103. The air-bag 107 is then positioned such that the two sets of tentacles 108 extend along the length of the splint members 102,103 and the umbilical 110 is located in the hinge region between the two splint members 102,103. Finally, the second pair of foam inserts 112,113 are positioned over the air-bag 108, which is thus interposed between the first and second pairs of foam inserts. The first pair of foam inserts 104,105 are secured to the splint members 102,103 and the second pair of foam inserts 112,113 to the first pair by any suitable means, eg adhesive, mechanical fastenings etc.

The second embodiment is used in a similar manner to the first. The splint members 102,103 are positioned around the injured wrist and fastened together using the straps 114,115. The air-bag 107 is then inflated through the inflation valve 111 until the splint 101 conforms precisely to the contour of the patient's arm and wrist. Any excess pressure, established during inflation or subsequently as a consequence of oedema, is released automatically by venting of air through the relief port 112.

1. An orthopaedic splint comprising at least one rigid member adapted to be secured to a human or animal ankle, wrist, knee or elbow to provide support thereto and to maintain the body parts to which it is secured in a constant mutual disposition, the rigid member being associated with an inflatable air-bag disposed, in use, between a greater part of the rigid member and the body part to which the splint is applied, the air-bag being fitted with valve means by which air can be introduced into the air-bag and retained therewithin and by which air is released from within the air-bag if the pressure of air within the air-bag exceeds a predetermined threshold as a result of swelling of the ankle, wrist, knee or elbow after the splint has been applied thereto.

2. A splint as claimed in claim 1, wherein the valve means comprises a single valve which is used for both inflation of the air-bag and release of pressure in the event that the internal pressure rises above the predetermined threshold.

3. A splint as claimed in claim 1, wherein separate valves are provided for inflation and for pressure-release.

4. A splint as claimed in any preceding claim, wherein the rigid member is formed in rigid synthetic plastics material.

5. A splint as claimed in any preceding claim, which comprises a pair of cooperating rigid members, each of generally hemi-cylindrical form, the two rigid members together defining a generally tubular casing that can be fitted about a limb or part of a limb.

6. A splint as claimed in any preceding claim, which comprises more than one rigid member, and the rigid members are secured together by releasable fastenings.

7. A splint as claimed in claim 6, wherein the releasable fastenings are of the hook-and-loop type with cooperating male and female components.

8. A splint as claimed in any preceding claim, which comprises a single airbag.

9. A splint as claimed in any preceding claim, which comprises a single airbag, the air-bag having the form of a plurality of tentacles or fingers connected at one end thereof and extending longitudinally along the interior of each of two splint members that together form, in use, a generally tubular casing.

10. A splint as claimed in any preceding claim, wherein the air-bag is formed of plastics material.

11. A splint as claimed in any preceding claim, wherein the air-bag is formed of elastic material.

12. A splint as claimed in any preceding claim, wherein an intermediate material is interposed between the air-bag and the patient's skin.

13. A splint as claimed in claim 12, wherein the intermediate material is a plastics foam material.

14. A splint as claimed in claim 13, wherein a second layer of plastics foam material is interposed between the air-bag and the internal surface of the rigid member.

15. A splint as claimed in any preceding claim, wherein one or more openings are provided in the rigid member to enhance flow of air to and from the patient's skin.

16. A splint as claimed in claim 1, wherein the rigid member is cast about a patient's limb, th air-bag being interposed between the limb and the material cast about it.
